(19) United States
(12) Patent Application Publication
(10) Pub. No.: US 2024/0049667 A1
(43) Pub. Date: Feb. 15, 2024

(54) Albugo-Candida-Resistant Brassica Oleracea Plants

(71) Applicant: Bejo Zaden B.V., Warmenhuizen (NL)

(72) Inventors: Johannes Theodorus Wilhelmus Ligthart, Warmenhuizen (NL); Jan Sybe Wijngaarden, Warmenhuizen (NL); Johannes Gerardus Maria Hoogland, Warmenhuizen (NL); Hubertus Theodorus Maria Janssen, Warmenhuizen (NL); Roelof Marinus Veenstra, Warmenhuizen (NL); Albertus Johannes Maria Schrijver, Warmenhuizen (NL)

(21) Appl. No.: 18/278,516

(22) PCT Filed: Feb. 24, 2021
(86) PCT No.: PCT/EP2021/054588

§ 371 Date: Aug. 23, 2023

(51) Int. Cl. A01H 1/00; C12Q 1/6895; A01H 6/20; A01H 1/02

(52) U.S. Cl. CPC A01H 1/1255; C12Q 1/6895; A01H 6/203; A01H 1/023; C12Q 2600/13

(57) ABSTRACT

Provided herein are Brassica oleracea plants being resistant to the plant pathogen Albugo candida and wherein the resistance is encoded by one genomic region on chromosome. Also provided herein are methods for identifying the present Albugo candida resistance and to molecular markers for use in the present methods.

### CROSS-REFERENCE TO RELATED APPLICATION

This application is the United States national phase of International Application No. PCT/EP2021/054588 filed Feb. 24, 2021, the disclosure of which is hereby incorporated by reference in its entirety.

The Sequence Listing associated with this application is filed in electronic format via EFS-Web and is hereby incorporated by reference into the specification in its entirety. The name of the text file containing the Sequence Listing is 2305748_ST25.txt. The size of the text file is 19,021 bytes, and the text file was created on Jul. 20, 2023.

### BACKGROUND OF THE INVENTION

### Field of the Invention

The present invention relates to Brassica oleracea plants being resistant to the plant pathogen Albugo candida and wherein the resistance is encoded by one genomic region on chromosome 2. The present invention further relates to methods for identifying the present Albugo candida resistance and to molecular markers for use in the present methods.

### Description of Related Art

Cabbage, or Brassica oleracea , is grown globally as a food crop. Almost every part of the B. oleracea plant is suitable for consumption. Several cultivars of B. oleracea exist, including headed cabbage, savoy cabbage, borecole and point headed cabbage (edible part: the leaves); broccoli, sprouting broccoli, Romanesco and cauliflower (edible part: the flower heads); Brussels sprouts (edible part: the lateral buds) and kohlrabi (edible part: the hypocotyl which looks like a thickened part of the stem of the plant). All of these vegetables are rich in essential nutrients, including vitamin C. A diet rich in cruciferous vegetables can reduce the risk of developing some types of human cancers.

As is the case for many cultivated crops, several diseases and pests pose a threat to the cultivation of B. oleracea. Among these is the oomycete Albugo candida , which causes a disease called white blister. This plant disease causes blisters with spores (sari, pustules) on the leaves, stems and ovaries (siliques) of Brassica plants. These blisters may merge together to form larger, irregular shaped lesions. Systemic infection of a plant results in abnormal growth, deformations and sometimes sterility of the flowers or inflorescence.

White blister or A. candida (other synonyms: A. cruciferum, A. cruciferatum, white rust, white blister rust, staghead) is an oomycete closely related to downy mildew (Peronospora parasitica) and Phytophthora.

The oomycete A. candida occurs in many parts of the world where plants belonging to the family of Brassicaceae (formerly referred to as Cruciferae) are grown, including Europe, Asia, Africa, Australasia, North, Central, and South America.

The spores of the oomycete are dispersed by wind, rain and insects to other plants, but also watering, farm equipment and farm workers can contribute to the spread of A. candida .

When spores of A. candida land on a Brassica plant, they form a germ tube with which they penetrate the leaf. After leaf penetration, the mycelium grows intercellularly and absorbs nutrients via haustoria. The mycelium also develops zoosporangia just beneath the epidermis of the host in which asexual spores called zoospores form. When there is enough moisture, the mature zoospores are released and spread to other plants to cause new infections. The spores have two whiplash tails (flagella), one to move forward and one to control swimming direction.

The oomycete A. candida thrives best at temperatures between and 10 and 20° C. and in moist conditions. A leaf wetness period of 2.5 hours is enough to result in infection with the first symptoms appearing after an incubation period of 10 to 14 days. Moist weather conditions with moderate temperatures are therefore ideal conditions for the disease to spread.

A. candida can overwinter in the ground in sexual form as thick-walled oospores on plant remnants, or in asexual form (mycelium) on winter-hardened host plants. During mild winters the oomycete does not become dormant but remains active at a lower level.

Besides B. oleracea, A. candida can also infect species related to B. oleracea, such as rape, mustard and radish, and wild species, such as shepherd's purse (Capsella bursa-pastoris) and wild mustard (charlock mustard, Sinapis arvensis).

Host specialization in A. candida is known and different physiological species and formae speciales are distinguished based on the plant species or the line that is infected and the aggressiveness of the isolate on this particular plant species or line.

Currently, only few agents can control white blister in Brassicas. Moreover, an increasing number of countries in Europe have a policy aimed at reducing the use of crop protection agents. If the use of control agents would no longer be allowed, this would lead to significant problems in the cultivation of Brassica crops. White blister can cause enormous losses in yield, especially in crops such as Brassica rapa (syn. campestris) (turnip rape), Brassica juncea (mustard) and Brassica napus (rapeseed). Moreover, in vegetable crops, like broccoli, Brussels sprouts, headed cabbage and curly kale, cosmetic damage caused by the infection will make the crop no longer marketable.

Considering the problems outlined above, it is a goal for (vegetable) plant breeding to develop resistant plants harbouring one or more resistance genes or genetic loci contributing to resistance to this pathogen. This approach also contributes to the more sustainable production of the crop involved. In general, resistance can be monogenic, i.e., determined by one locus or gene, or depend on several loci or genes. In the latter case, these genes can be additive, resulting in Quantitative Trait Loci or QTLs.

The availability of marker sequences linked to the resistance gene or genes contributes to the acceleration of the breeding process as B. oleracea is a biannual crop Linking specific DNA markers to a resistance gene makes it possible to identify resistant plants in the offspring of various crosses. The use of DNA markers allows the researcher to directly test the seedling for the presence of a particular resistance without the need for time-consuming field tests. As a result, the biannual life cycle of B. oleracea no longer limits the ability of the researcher to test for resistance to A. candida . Hence, the use of DNA markers to select for desirable traits referred to as marker-assisted breeding makes it possible to rapidly introduce a resistance gene from one parental line to several B. oleracea crops.

In general, breeding for resistance starts by making a cross between a source of resistance and susceptible genetic material with a high level of agronomical quality. Resistant offspring is selected using DNA markers and repeatedly backcrossed to the agronomically elite parent line. This process ultimately leads to resistant plants with desirable agronomic characteristics. Application of cell biological techniques, such as doubled haploid induction (anther culture or microspore culture), can accelerate breeding by giving a high level of genetic purity within one generation.

### SUMMARY OF THE INVENTION

Considering the above, it is an object of the present invention, amongst others, to provide novel Albugo candida -resistance-providing genomic fragments and plants comprising these fragments.

The present invention meets the above object, amongst other objects, as outlined in the appended claims.

Specifically, this object, amongst other objects, is achieved by providing Brassica oleracea plants wherein the plants are resistant to the plant pathogen Albugo candida , and wherein the resistance is encoded by one genomic region located on chromosome 2 between base pairs 5373001 and 6058829.

### DESCRIPTION OF THE INVENTION

Although the present genomic fragment can be introduced into Brassica oleracea plants by introgression, the genomic fragment can be artificially introduced in plant cells to generate Albugo candida-resistant plants using various genome engineering techniques.

As the genomic region is known, the genomic fragment can, for example, be transferred between plants using microplast-mediated chromosome transfer. Using this method, entire chromosomes or parts thereof can be horizontally transferred between plants. First, micro-protoplasts containing one or a few chromosomes that carry the resistance are generated. Subsequently, the micro-protoplasts are fused with protoplasts generated from a susceptible Brassica oleracea plant. This method produces plants with monosomic additions, which can subsequently be crossed with other plants to generate Albugo candida -resistant lines.

Alternatively, as the nucleotide sequences of the present genomic fragment is known, these fragments can also be artificially assembled in yeast and subsequently allowed to recombine with the Brassica oleracea genome. Sections of the genomic fragment can also be amplified by long-range PCR amplifications or de novo synthesized and the resulting fragments reassembled and transformed into Brassica oleracea cells in a single step or in a series of transformations ultimately resulting in the present Brassica oleracea plants. The present genomic fragment, completely or in parts later to be reassembled, can also be isolated from gels or columns, for example, after restriction digestion, and subsequently transformed into Brassica oleracea cells.

Yet alternatively, the genomic fragment of interest can be introduced into a vector under a (strong) promotor. Subsequently, susceptible plants can be transformed with the vector and the sequence of interest expressed resulting in resistance. These techniques are readily available for the skilled person. Construction of artificial chromosomes comprising the present genomic fragments is also contemplated within the context of the present invention.

According to a preferred embodiment of the present invention, the present genomic region is obtainable, obtained, or is from a Brassica oleracea plant resistant to Albugo candida comprising one genomic region located on chromosome 2 from base pairs 5373001 to 6058829 deposited at NCIMB (National Collections of Industrial, Food and Marine Bacteria; NCIMB Limited, Ferguson Building; Craibstone Estate, Bucksburn Aberdeen, Scotland, AB21 9YA United Kingdom) on 6 Aug. 2019 under number NCIMB 43452.

The present Brassica oleracea plants preferably comprise one or more genomic sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, 30 SEQ ID No. 21, SEQ ID No. 23, SEQ ID No. 25, SEQ ID No. 27, SEQ ID No. 29, SEQ ID No. 31, SEQ

ID No. 33, SEQ ID No. 35, SEQ ID No. 37, SEQ ID No. 39, SEQ ID No. 41, and SEQ ID No. 43. The odd SEQ ID numbers represent the sequences corresponding to the resistance allele, while the even SEQ ID numbers represent the sequences corresponding to the susceptible allele. Hence, SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28, SEQ ID No. 30, SEQ ID No. 32, SEQ ID No. 34, SEQ ID No. 36, SEQ ID No. 38, SEQ ID No. 40, SEQ ID No. 42, and SEQ ID No. 44 represent the sequences corresponding to the susceptible allele.

According to a preferred embodiment, the present Brassica oleracea plants are cytoplasmic male sterile (CMS).

According to yet another preferred embodiment, the present Brassica oleracea plants are hybrid plants.

Preferably, the present Brassica oleracea plants are selected from the group consisting of Brassica oleracea convar. botrytis var. botrytis (cauliflower, Romanesco), Brassica oleracea convar. botrytis var. cymosa (broccoli), Brassica oleracea convar. botrytis var. asparagoides (sprouting broccoli),

Brassica oleracea convar. oleracea var. gemnifera (Brussels sprouts), Brassica oleracea convar. capitata var. alba (white cabbage, oxheart cabbage), Brassica oleracea convar. capitata var. rubra (red cabbage), Brassica oleracea convar. capitata var. sabauda (savoy cabbage), Brassica oleracea convar. acephela var. sabellica (curly kale cabbage), Brassica oleracea convar. acephela var. gongylodes (turnip cabbage) and Brassica oleracea var. tronchuda syn. costata (Portuguese cabbage).

The present invention also relates to hybrid Brassica oleracea plants obtainable either by crossing Albugo candida -susceptible Brassica oleracea plants with Brassica oleracea plants comprising the present Albugo candida resistance or by crossing an Albugo candida -susceptible Brassica olereacea plant with deposit NCIMB 43452.

According to an especially preferred embodiment of the present invention, the present resistance providing genomic fragment is obtainable, obtained or derived from a Brassica plant of which representative seeds are deposited under NCIMB 43452 on 6 August 2019 at the NCIMB (NCIMB Limited, Ferguson Building; Craibstone Estate, Bucksburn ABERDEEN, Scotland, AB21 9YA United Kingdom).

Within the context of the present invention the following B. oleracea plant are contemplated. B. oleracea convar. botrytis var. botrytis (cauliflower, Romanesco), B. oleracea convar. botrytis var. cymosa (broccoli), B. oleracea convar. botrytis var. asparagoides (sprouting broccoli), B. oleracea convar. oleracea var. gemnifera (Brussels sprouts), B. oleracea convar. capitata var. alba (white cabbage, point headed cabbage), B. oleracea convar. capitata var. rubra (red cabbage), B. oleracea convar. capitata var. sabauda (savoy cabbage), B. oleracea convar. acephala var. sabellica (borecole), B. oleracea convar. acephela var. gongylodes (kohlrabi) and B. oleracea var. tronchuda syn. costata (Portuguese cabbage).

The present invention further relates to methods for identifying the genomically-encoded resistance against the plant pathogen Albugo candida as found in the Brassica oleracea plant deposited under deposit number NCIMB 43452, the method comprises the step of detecting the presence of one or more genomic sequences selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39, SEQ ID No. 41, and SEQ ID No. 43.

The present invention further also relates to seeds or plant parts of plants defined above or to seeds capable of providing the present plants and to molecular markers which markers co-segregate with the genomically-encoded resistance against the plant pathogen Albugo candida as present in deposit NCIMB 43452.

The present invention furthermore relates to molecular markers which markers co-segregate with a genomically encoded resistance against the plant pathogen Albugo candida as present in deposit NCIMB 43452, which molecular markers are selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No.

27, SEQ ID No. 29, SEQ ID No. 31, SEQ ID No. 33, SEQ ID No. 35, SEQ ID No. 37, SEQ ID No. 39, SEQ ID No. 41, and SEQ ID No. 43.

The present invention will be further detailed in the following examples.

### EXAMPLES

### Example 1.

### Populations and Disease Test

The white blister resistance originates from the parent line 947354 of Bejo Zaden B.V. of which seeds were deposited at the NCIMB (NCIMB Limited, Ferguson Building; Craibstone Estate, Bucksburn ABERDEEN, Scotland, AB21 9YA, United Kingdom) on 6 Aug. 2019 under number NCIMB 43452.

This source was crossed with different B. oleracea species (curly kale, cabbage, turnip cabbage, broccoli, sprouting broccoli, white cabbage, oxheart cabbage, red cabbage, savoy cabbage, tronchuda, Brussels sprouts and cauliflower). BC1 populations were obtained after backcrossing with susceptible parent lines. Resistant plants were selected from these populations using a disease test.

Isolates of A. candida were obtained by isolating zoosporangia from susceptible B. oleracea plants in the field. After germination in water, the spores were used to inoculate susceptible plants. After the development of blisters, these zoosporangia were harvested and stored in liquid nitrogen until use.

The disease test took place in a glasshouse on seedlings of the BC1 population 24 to 48 hours after development of the seed leaves. The plants were inoculated with a fresh zoospore suspension 5×10⁴ zoospores per ml) which was prepared by washing zoosporangia from susceptible plants and allowing them to germinate in water. Several drops of zoospore suspension were pipetted onto the seed leaves. After this procedure, the plants were grown under a plastic tunnel to guarantee optimal conditions for infection. Two weeks after inoculation, the plants were assessed by grouping them in three classes: resistant, susceptible or intermediate. After performing the disease test on the seedlings, the resistant plants were retained for the backcrossing program.

The results of the disease test showed that the resistance was, in principle, a monogenic dominant trait. Plants with intermediate reactions were, however, also often found in addition to susceptible and resistant plants. The presence of plants with an intermediate resistance was found to be highly dependent on the genetic background of the plants. Several populations were selected for the breeding program that had no, or hardly any, intermediate resistance and in which the expected segregation ratio (1:1 for a BC and 3:1 for self-pollination) was found.

### Example 2.

### Molecular Characterization of Genomic DNA and Mapping of the Resistance Gene

Several backcross populations were produced by crossing and repeated backcrossing of the source of resistance, deposited as NCIMB 43452 and a variety of B. oleracea cultivars. A set of SNP markers was subsequently developed by comparing sequence data from lines susceptible and resistant to A. candida . These SNP markers were repeatedly mapped on different Brassica populations. By selecting crossovers, the mapped region was narrowed down to the markers listed in Table 1.

The analysis of several generations of plants made it possible to reduce the genetic location of the resistance gene to an area of ˜465.000 bp, which corresponds to approx. 0.7% of this chromosome. Many SNP markers are in this area, enabling precise and rapid identification of plants harbouring the gene resulting in resistance to A. candida .

The locus defining A. candida resistance was determined to be on chromosome 2, and the positions of the SNP markers developed are found in Table 2. Abbreviations are according to IUPAC nucleotide code:

| Symbol | Nucleotide Base |
|---|---|
| A | Adenine |
| C | Cytosine |
| G | Guanine |
| T | Thymine |
| N | A or C or G or T |
| M | A or C |
| R | A or G |
| W | A or T |
| S | C or G |
| Y | C or T |
| K | G or T |
| V | Not T |
| H | Not G |
| D | Not C |
| B | Not A |

TABLE 1
SNPs for the detection of resistance against A. candida.
The reference genome was the updated assembly of the Brassica oleracea
reference genome, JZS v2 (Cai et al., Improved Brassica oleracea
JZS assembly reveals significant changing of LTR-RT dynamics
in different morphotypes, Theoretical and Applied Genetics
2020).

| | | Position on | Allele | |
|---|---|---|---|---|
| | | Chromosome 2 | linked to | Alternative |
| SNP | KSNP | (bp) | resistance | allele |
| 1 | 1009-4271.1 | 5373001 | T | C |
| 2 | 1009-4273.1 | 5385215 | A | G |
| 3 | 1009-4281.1 | 5697266 | T | G |
| 4 | 1009-4294.1 | 5453680 | C | G |
| 5 | 1009-2712.1 | 5455211 | T | C |
| 6 | 1009-0673.1 | 5481017 | T | C |
| 7 | 1009-0672.1 | 5480996 | C | T |
| 8 | 1009-2710.1 | 5487235 | A | C |
| 9 | 1009-2709.1 | 5514066 | G | A |
| 10 | 1009-2707.1 | 5518162 | T | C |
| 11 | 1009-0106.1 | 5559368 | T | A |
| 12 | 1009-0663.1 | 5559789 | A | G |
| 13 | 1009-2705.1 | 5573298 | A | G |
| 14 | 1009-6115.1 | 5740881 | G | T |
| 15 | 1009-6153.1 | 5750175 | A | G |
| 16 | 1009-6154.1 | 5766914 | T | G |
| 17 | 1009-6199.1 | 5776195 | G | C |
| 18 | 1009-6155.1 | 5791347 | C | T |
| 19 | 1009-6157.1 | 5840760 | A | G |
| 20 | 1009-6161.1 | 5933093 | A | C |
| 21 | 1009-2703.1 | 6007107 | C | T |
| 22 | 1009-2701.1 | 6058829 | G | A |

TABLE 2
Sequence and position on chromosome 2 of SNPs used for the detection of
resistance against A. candida. Sequences with odd numbers are linked to
resistance to A. candida, whereas sequences with even numbers to
susceptibility. The reference genome was the updated assembly of the
Brassica oleracea reference genome, JZS v2 (Cai et al., Improved
Brassica oleracea JZS assembly reveals significant changing of
LTR-RT dynamics in different morphotypes, Theoretical and Applied
Genetics 2020).

| SEQ | Position | |
|---|---|---|
| ID | on Chr 2 | Sequence |
| No. | (bp) | (SNP nucleotide is bold and in brackets) |
| 1 | 5373001 | AAAAAATATGGAGTGAAATACAAAGATTAAATTAATAAATAGAATGA |
| | | AACAATAAAGATTCAGACCAAAACCTATCAACCAACTAAGCAACCAG |
| | | ACATGC[T]MGAACMARAAAAATYGRGGATAGTCGAAGTCRARAACAA |
| | | TGCAHCACAATACCGAGARAWAAKTGTTCTCAAACCTTGAAACAAYTC |
| | | CTTCTACAGCYKC |
| 2 | 5373001 | AAAAAATATGGAGTGAAATACAAAGATTAAATTAATAAATAGAATGA |
| | | AACAATAAAGATTCAGACCAAAACCTATCAACCAACTAAGCAACCAG |
| | | ACATGC[C]MGAACMARAAAAATYGRGGATAGTCGAAGTCRARAACAA |
| | | TGCAHCACAATACCGAGARAWAAKTGTTCTCAAACCTTGAAACAAYTC |
| | | CTTCTACAGCYKC |
| 3 | 5385215 | ATCGAATAATGTAATTTGTATTTTTATAAATTTAATTTCACTCAATAYA |
| | | YATATATATGATATAGTCATATAGACGTGGYTTGGCAGAAAAAGAKGG |
| | | AGA[A]CACACTCATGGTTWATAGAAAAAGAGGGAACAAAGTAATAGC |
| | | GAGGTTGTCCYWTTCTTCTTGATCARTGATTATSRATCKGTTTCGTAGT |
| | | GCTCTTGTT |
| 4 | 5385215 | ATCGAATAATGTAATTTGTATTTTTATAAATTTAATTTCACTCAATAYA |
| | | YATATATATGATATAGTCATATAGACGTGGYTTGGCAGAAAAAGAKGG |
| | | AGA[G]CACACTCATGGTTWATAGAAAAAGAGGGAACAAAGTAATAGC |
| | | GAGGTTGTCCYWTTCTTCTTGATCARTGATTATSRATCKGTTTCGTAGT |
| | | GCTCTTGTT |
| 5 | 5697266 | CATATCATAAAAGCTAATGGAAGTAAATGGGAACSAACCATCTSCGAG |
| | | ARTCATAACCAGCTATATTGGCGACACCCTCCAAAGCTTCCCTCCATGC |
| | | CTT[T]ACCTTTTCTTCTTTCCCCACAGGTTTTTTCAAAGGCTTTCCCG |
| | | AAATCTCCGGTCTGCTTCCTAACMTCAGATGGATCCACTTCGTAGAAAA |
| | | TGGATATC |
| 6 | 5697266 | CATATCATAAAAGCTAATGGAAGTAAATGGGAACSAACCATCTSCGAG |
| | | ARTCATAACCAGCTATATTGGCGACACCCTCCAAAGCTTCCCTCCATGC |
| | | CTT[G]ACCTTTTCTTCTTTCCCCACAGGTTTTTTCAAAGGCTTTCCCG |
| | | AAATCTCCGGTCTGCTTCCTAACMTCAGATGGATCCACTTCGTAGAAAA |
| | | TGGATATC |
| 7 | 5453680 | AAAAACAAATACAAGAAATGTACCAACTGTTAAGCCAAGAAATCTGA |
| | | GAACACATAATGTCAGAGGCTCAGAGCACGAGCACGAGTATTTCACAT |
| | | AACTA[C]AAGATGGTGTTAAAAGATTTACCAAAATAAATGCATTTGGC |
| | | ATATACGGAAGGAATAATTAGAAATACAAATCTAAGAAATTTATTTGA |
| | | GTTRAMAAAAA |
| 8 | 5453680 | AAAAACAAATACAAGAAATGTACCAACTGTTAAGCCAAGAAATCTGA |
| | | GAACACATAATGTCAGAGGCTCAGAGCACGAGCACGAGTATTTCACAT |
| | | AACTA[G]AAGATGGTGTTAAAAGATTTACCAAAATAAATGCATTTGGC |
| | | ATATACGGAAGGAATAATTAGAAATACAAATCTAAGAAATTTATTTGA |
| | | GTTRAMAAAAA |
| 9 | 5455211 | AACTTGAGTTATTTCATTCTCATGTACTCGAACACATACATCTTGAGAA |
| | | CTGAATAATATAGTATAAACGAATAAAACTGAACTTAGGGATTGCTCA |
| | | AAC[T]GAGTTTCCCACTTCATCATGTGTGGCTCATAGGGCAAGAGCAG |
| | | AGCTAAGGTTCATAGGGTTCATATACTTGGTGGTACCGGTCAATATATG |
| | | ACGGACTA |
| 10 | 5455211 | AACTTGAGTTATTTCATTCTCATGTACTCGAACACATACATCTTGAGAA |
| | | CTGAATAATATAGTATAAACGAATAAAACTGAACTTAGGGATTGCTCA |
| | | AAC[C]GAGTTTCCCACTTCATCATGTGTGGCTCATAGGGCAAGAGCAG |
| | | AGCTAAGGTTCATAGGGTTCATATACTTGGTGGTACCGGTCAATATATG |
| | | ACGGACTA |
| 11 | 5481017 | ACCTCCTCGCTGATGACCTTTTCGAGAATCATCCAAGGAGGATGACTCT |
| | | GTATGAACTGACAGTTTCTTTCCATGTTGATGCACCGAAAACAAGAAG |
| | | CAACCAAACAAAAGAAAGAAGATTGTAAAAGTCCATTCRTACACCAA |
| | | GATCAAACCAGTCCATGGCATGATTTGCCTCGGCAYAATCACAAAGGA |
| | | AGTTCCAA[T]GGATATCAGAAGTGCAGTAAAACAGACTAGAACTGAA |
| | | ACTGCGCCTAAGCGCTGAGGAACTTTGGAGTGTATGCTGCCACTGTGG |
| | | AGTTGATAGCTGGGATACATGGTTGAAAATGTAGAAACACCGCGTGTT |
| | | CCATTAGATCTGATTCTGTAATAAAGATATCTAATCTGATTGAATAATG |
| | | AACCCTCATGAACCTGAA |
| 12 | 5481017 | ACCTCCTCGCTGATGACCTTTTCGAGAATCATCCAAGGAGGATGACTCT |
| | | GTATGAACTGACAGTTTCTTTCCATGTTGATGCACCGAAAACAAGAAG |
| | | CAACCAAACAAAAGAAAGAAGATTGTAAAAGTCCATTCRTACACCAA |
| | | GATCAAACCAGTCCATGGCATGATTTGCCTCGGCAYAATCACAAAGGA |
| | | AGTTCCAA[C]GGATATCAGAAGTGCAGTAAAACAGACTAGAACTGAA |
| | | ACTGCGCCTAAGCGCTGAGGAACTTTGGAGTGTATGCTGCCACTGTGG |
| | | AGTTGATAGCTGGGATACATGGTTGAAAATGTAGAAACACCGCGTGTT |
| | | CCATTAGATCTGATTCTGTAATAAAGATATCTAATCTGATTGAATAATG |
| | | AACCCTCATGAACCTGAA |
| 13 | 5480996 | TGTAGTAACGTCACAAGACACACCTCCTCGCTGATGACCTTTTCGAGA |
| | | ATCATCCAAGGAGGATGACTCTGTATGAACTGACAGTTTCTTTCCATGT |
| | | TGATGCACCGAAAACAAGAAGCAACCAAACAAAAGAAAGAAGATTGT |
| | | AAAAGTCCATTCRTACACCAAGATCAAACCAGTCCATGGCATGATTTG |
| | | CCTCGGCA[C]AATCACAAAGGAAGTTCCAAYGGATATCAGAAGTGCA |
| | | GTAAAACAGACTAGAACTGAAACTGCGCCTAAGCGCTGAGGAACTTTG |
| | | GAGTGTATGCTGCCACTGTGGAGTTGATAGCTGGGATACATGGTTGAA |
| | | AATGTAGAAACACCGCGTGTTCCATTAGATCTGATTCTGTAATAAAGA |
| | | TATCTAATCTGATTGAATA |
| 14 | 5480996 | TGTAGTAACGTCACAAGACACACCTCCTCGCTGATGACCTTTTCGAGA |
| | | ATCATCCAAGGAGGATGACTCTGTATGAACTGACAGTTTCTTTCCATGT |
| | | TGATGCACCGAAAACAAGAAGCAACCAAACAAAAGAAAGAAGATTGT |
| | | AAAAGTCCATTCRTACACCAAGATCAAACCAGTCCATGGCATGATTTG |
| | | CCTCGGCA[T]AATCACAAAGGAAGTTCCAAYGGATATCAGAAGTGCAG |
| | | TAAAACAGACTAGAACTGAAACTGCGCCTAAGCGCTGAGGAACTTTGG |
| | | AGTGTATGCTGCCACTGTGGAGTTGATAGCTGGGATACATGGTTGAAA |
| | | ATGTAGAAACACCGCGTGTTCCATTAGATCTGATTCTGTAATAAAGAT |
| | | ATCTAATCTGATTGAATA |
| 15 | 5487235 | TCAAGAACGACCATCCCGTTCCGATCAAGATGATCACGGTGAAAAGCA |
| | | ACACGACACGAATGAATTGGAAGATGTAGAAGAGGATGTCCCATCCGT |
| | | GAGG[A]GTCCCCGTGATCTTCACGTARTGCTTATCYTCAGCTGCGCAGA |
| | | TCAGATTCAAAGACTTGATTAAAAGCAGACCCGCCATGAGGAGATGGA |
| | | TCC |
| 16 | 5487235 | TCAAGAACGACCATCCCGTTCCGATCAAGATGATCACGGTGAAAAGCA |
| | | ACACGACACGAATGAATTGGAAGATGTAGAAGAGGATGTCCCATCCGT |
| | | GAGG[C]GTCCCCGTGATCTTCACGTARTGCTTATCYTCAGCTGCGCAGA |
| | | TCAGATTCAAAGACTTGATTAAAAGCAGACCCGCCATGAGGAGATGGA |
| | | TCC |
| 17 | 5514066 | GAGATGGAGTTGGTGTGGCATGACTCAGCCAATGGYTCGAGCCGTCCT |
| | | ACAAATTCGAACAAGACTTCYACAGACTCAGTTAGATGGCCTCAATGG |
| | | AAGT[G]AACCAACMGAGAAGTGAATATGATTACGTTTCCGGTTCAGTG |
| | | GATTAACCAACAGGTTGCAGATCATTGAATCGATATGTTTGTATGTTTA |
| | | AATATAATA |
| 18 | 5514066 | GAGATGGAGTTGGTGTGGCATGACTCAGCCAATGGYTCGAGCCGTCCT |
| | | ACAAATTCGAACAAGACTTCYACAGACTCAGTTAGATGGCCTCAATGG |
| | | AAGT[A]AACCAACMGAGAAGTGAATATGATTACGTTTCCGGTTCAGTG |
| | | GATTAACCAACAGGTTGCAGATCATTGAATCGATATGTTTGTATGTTTA |
| | | AATATAATA |
| 19 | 5518162 | GTTTCTATAAGAAGAAACCAGAAGAAGGGTCTATTAGTGGAAGGGTCC |
| | | AGAGGCTTGCDAAGTATCGATTCTTGAAGAAACAATCGGATCTKTTGT |
| | | TGAA[T]TCTGATGATTTGGCTGCTATGTGGAATTGTCTGAGAGAAAATT |
| | | GTGTGATTGATGATGCCACTGGTGCTGAAAAGATGAACTATGAAGACT |
| | | TCTGCCACA |
| 20 | 5518162 | GTTTCTATAAGAAGAAACCAGAAGAAGGGTCTATTAGTGGAAGGGTCC |
| | | AGAGGCTTGCDAAGTATCGATTCTTGAAGAAACAATCGGATCTKTTGT |
| | | TGAA[C]TCTGATGATTTGGCTGCTATGTGGAATTGTCTGAGAGAAAATT |
| | | GTGTGATTGATGATGCCACTGGTGCTGAAAAGATGAACTATGAAGACT |
| | | TCTGCCACA |
| 21 | 5559368 | TCACGCATGACCATGATATTGTTCCTCATCTGCCTCCTTACTACAACCA |
| | | TTTTCCTCAAAAAACATACCACCACTTCCCAACAGAGGTGTGGCTAAG |
| | | AGATGTC[T]GTTCCTTGAATCATAGTGTGGAGAAAGTTTGTGACAACA |
| | | CCGGTGAAGATCCAACATGCAGCAGGTCGGTGAAGGGCAATAGCATTT |
| | | CAGACCATCTAAGGTACTTTGGGGTAGAGTTGCATTGTGAGACTTGGA |
| | | GACAATGCTCAATAGTGATGAGCCATGAGATGGATAGATTCAGCAAGA |
| | | AGGATTCAAAGGGTAAT |
| 22 | 5559368 | TCACGCATGACCATGATATTGTTCCTCATCTGCCTCCTTACTACAACCA |
| | | TTTTCCTCAAAAAACATACCACCACTTCCCAACAGAGGTGTGGCTAAG |
| | | AGATGTC[A]GTTCCTTGAATCATAGTGTGGAGAAAGTTTGTGACAACA |
| | | CCGGTGAAGATCCAACATGCAGCAGGTCGGTGAAGGGCAATAGCATTT |
| | | CAGACCATCTAAGGTACTTTGGGGTAGAGTTGCATTGTGAGACTTGGA |
| | | GACAATGCTCAATAGTGATGAGCCATGAGATGGATAGATTCAGCAAGA |
| | | AGGATTCAAAGGGTAAT |
| 23 | 5559789 | CATAGTGTGGAGAAAGTTTGTGACAACACCGGTGAAGATCCAACATGC |
| | | AGCAGGTCGGTGAAGGGCAATAGCATTTCAGACCATCTAAGGTACTTT |
| | | GGGGTAGAGTTGCATTGTGAGACTTGGAGACAATGCTCAATAGTGATG |
| | | AGCCATGAGATGGATAGATTCAGCAAGAAGGATTCAAAGGGTAATCTA |
| | | ATCATGTC[A]CGGAATGTTCCTTCCACCAACGGTAACAAAACAGAATC |
| | | TCTTATCGAAAATGGGGATCTTTAGTCTATAGGAATCGTTGATTCAAGT |
| | | CTTGGTCAAGCAAAGCTTGCTTCAAAAGGAGATTCCGGTGTTGGAGAA |
| | | AGAAAGAAAGTGTATAGATACATATAATCAAGACTTTGTAAATAGGTT |
| | | GTAGGTTGATAGTACGT |
| 24 | 5559789 | CATAGTGTGGAGAAAGTTTGTGACAACACCGGTGAAGATCCAACATGC |
| | | AGCAGGTCGGTGAAGGGCAATAGCATTTCAGACCATCTAAGGTACTTT |
| | | GGGGTAGAGTTGCATTGTGAGACTTGGAGACAATGCTCAATAGTGATG |
| | | AGCCATGAGATGGATAGATTCAGCAAGAAGGATTCAAAGGGTAATCTA |
| | | ATCATGTC[G]CGGAATGTTCCTTCCACCAACGGTAACAAAACAGAATC |
| | | TCTTATCGAAAATGGGGATCTTTAGTCTATAGGAATCGTTGATTCAAGT |
| | | CTTGGTCAAGCAAAGCTTGCTTCAAAAGGAGATTCCGGTGTTGGAGAA |
| | | AGAAAGAAAGTGTATAGATACATATAATCAAGACTTTGTAAATAGGTT |
| | | GTAGGTTGATAGTACGT |
| 25 | 5573298 | CCTTTGTACTAAACCACTTAATGGCACAGTGCTCATGAACGAGCCTGA |
| | | GGTCACCTTTGCAACTGCATTCCATTTTCAACGTGTTGCCTTCCTCGCA |
| | | GAC[A]TCAAGACAAATCCTGCACACCGCTTCTTCTTCAGGGATCTCTTC |
| | | TTCAGTTTCTTCCGCAGTAACCGGAGTGATTTCATCTCCACAACCACTT |
| | | GCTTCAT |
| 26 | 5573298 | CCTTTGTACTAAACCACTTAATGGCACAGTGCTCATGAACGAGCCTGA |
| | | GGTCACCTTTGCAACTGCATTCCATTTTCAACGTGTTGCCTTCCTCGCA |
| | | GAC[G]TCAAGACAAATCCTGCACACCGCTTCTTCTTCAGGGATCTCTTC |
| | | TTCAGTTTCTTCCGCAGTAACCGGAGTGATTTCATCTCCACAACCACTT |
| | | GCTTCAT |
| 27 | 5740881 | TTAGGTGTCAGGTCCYGGGTTGTGAAGTGGATATAAGCGAGCTCAAAG |
| | | GGTAYCATARAAGGCATAGGGTTTGYCTCACGTGTGCTAACGCTAGCT |
| | | CCGT[G]GTGCTTGAGGGAGTGGATAAGAGATACTGTCAACAGTGTGGA |
| | | AAGTAWGTTCCTTTTATTGTTAATTTGATCCTATGCTTTATGGCTTAAC |
| | | AGATACATA |
| 28 | 5740881 | TTAGGTGTCAGGTCCYGGGTTGTGAAGTGGATATAAGCGAGCTCAAAG |
| | | GGTAYCATARAAGGCATAGGGTTTGYCTCACGTGTGCTAACGCTAGCT |
| | | CCGT[T]GTGCTTGAGGGAGTGGATAAGAGATACTGTCAACAGTGTGGA |
| | | AAGTAWGTTCCTTTTATTGTTAATTTGATCCTATGCTTTATGGCTTAAC |
| | | AGATACATA |
| 29 | 5750175 | TCAACAGTCTCAACTCTACGGTTCAAACACCTGAATCTCAGTTTGTGCA |
| | | CCGGTTGCTCGACAGACTACATGCTCTCCATCAGGATCACATGAGCTA |
| | | CAA[A]CATGTGGTTGAAAAGCCTTTTAGTTTTCCGCTTCCTAATAARGA |
| | | TGATCTTGTCTGGTTTTTAAACAAACCCTTTTAACTGTTGTTCCAGGGG |
| | | ATGTTCT |
| 30 | 5750175 | TCAACAGTCTCAACTCTACGGTTCAAACACCTGAATCTCAGTTTGTGCA |
| | | CCGGTTGCTCGACAGACTACATGCTCTCCATCAGGATCACATGAGCTA |
| | | CAA[G]CATGTGGTTGAAAAGCCTTTTAGTTTTCCGCTTCCTAATAARGA |
| | | TGATCTTGTCTGGTTTTTAAACAAACCCTTTTAACTGTTGTTCCAGGGG |
| | | ATGTTCT |
| 31 | 5766914 | AACCATAATCTGGAGAMTTTTGACCAAAAGCATATTGACASAAGATCT |
| | | GCAGAGCCCAAGTTGAAGCTGGAAATATCATCTCATACATATGGTTGG |
| | | TCCY[T]AGTCCCAGTGACTTGAGAAGTTTTTTATCTTCGGTTGTAATGA |
| | | TAACAATACTTCCCGGACCAACCCATCCACGCTGGTTTGCCATCTCCTC |
| | | TAATTGYC |
| 32 | 5766914 | AACCATAATCTGGAGAMTTTTGACCAAAAGCATATTGACASAAGATCT |
| | | GCAGAGCCCAAGTTGAAGCTGGAAATATCATCTCATACATATGGTTGG |
| | | TCCY[G]AGTCCCAGTGACTTGAGAAGTTTTTTATCTTCGGTTGTAATGA |
| | | TAACAATACTTCCCGGACCAACCCATCCACGCTGGTTTGCCATCTCCTC |
| | | TAATTGYC |
| 33 | 5776195 | TTTGAATTCCACAAGATTAGCTATACARYATTACTTTTTGAAACTAAAC |
| | | TAAGTTATATTGTAACGCATGACSGGCTACAGYTAATGGACTTTCCACG |
| | | CT[G]ACTCACTCKGTTGGTGTGCTTCATATGCGTGCGCATGGCGGTATA |
| | | TTAATTTTTTGGAGGCTCCTARGACTTGTYTATTAACTCTTAATCAACC |
| | | ACRTRA |
| 34 | 5776195 | TTTGAATTCCACAAGATTAGCTATACARYATTACTTTTTGAAACTAAAC |
| | | TAAGTTATATTGTAACGCATGACSGGCTACAGYTAATGGACTTTCCACG |
| | | CT[C]ACTCACTCKGTTGGTGTGCTTCATATGCGTGCGCATGGCGGTATA |
| | | TTAATTTTTTGGAGGCTCCTARGACTTGTYTATTAACTCTTAATCAACC |
| | | ACRTRA |
| 35 | 5791347 | CGAGGAGTTGTACTTTTTTCTTTGTAAACAATATTTGCTTGCGCAATAA |
| | | ATTGAACATTCCCGAAAATAACCTATCGCTTTTACCCCTAAAAAAAATT |
| | | AC[C]GCCAAAAAGTTGAAGCATGACATATTTAGGTCCGAGTCTTCTTCT |
| | | TCGTCTCAATATATATTGTGGGGCCAGCAATTTGGTGGGAACCGTCGA |
| | | CGTGGAA |
| 36 | 5791347 | CGAGGAGTTGTACTTTTTTCTTTGTAAACAATATTTGCTTGCGCAATAA |
| | | ATTGAACATTCCCGAAAATAACCTATCGCTTTTACCCCTAAAAAAAATT |
| | | AC[T]GCCAAAAAGTTGAAGCATGACATATTTAGGTCCGAGTCTTCTTCT |
| | | TCGTCTCAATATATATTGTGGGGCCAGCAATTTGGTGGGAACCGTCGA |
| | | CGTGGAA |
| 37 | 5840760 | ACCCCAACACATTGCCTTGATGTTGAAATTAATTAATCACTATCCGTGT |
| | | TCARTATTGTCTCTCCAGSCAAGTAAGTATTTGATTTTAATCATACTTTA |
| | | A[A]TTTACAYTGCTCTTGGCCGCCTAGAAGAAACATAACAATTCAGGC |
| | | CTTTGATCTTGACCYCGTTCGAAAATAGGCTCTTCTGCTGTGAACCAAA |
| | | GGAGTA |
| 38 | 5840760 | ACCCCAACACATTGCCTTGATGTTGAAATTAATTAATCACTATCCGTGT |
| | | TCARTATTGTCTCTCCAGSCAAGTAAGTATTTGATTTTAATCATACTTTA |
| | | A[G]TTTACAYTGCTCTTGGCCGCCTAGAAGAAACATAACAATTCAGGC |
| | | CTTTGATCTTGACCYCGTTCGAAAATAGGCTCTTCTGCTGTGAACCAAA |
| | | GGAGTA |
| 39 | 5933093 | TGCCTCGATCTTGACATRARCTATATTGATGTCTGTCAGATTCTTTGTGT |
| | | ATTCATCTGTCTYCTTARGCTCACCAATCAACCCAGSAGCRAAGCTTMG |
| | | A[A]CTTCAAGGCTACGCAAGTTGAGAGGAAGACCAATCAAGTGAGCCC |
| | | ACAKAGGGATCGACTCCATATCTGGAGTGGAGGCCTCGTGCTTGGAGG |
| | | TCAACGR |
| 40 | 5933093 | TGCCTCGATCTTGACATRARCTATATTGATGTCTGTCAGATTCTTTGTGT |
| | | ATTCATCTGTCTYCTTARGCTCACCAATCAACCCAGSAGCRAAGCTTMG |
| | | A[C]CTTCAAGGCTACGCAAGTTGAGAGGAAGACCAATCAAGTGAGCCC |
| | | ACAKAGGGATCGACTCCATATCTGGAGTGGAGGCCTCGTGCTTGGAGG |
| | | TCAACGR |
| 41 | 6007107 | ATTCACGAGCAGCTTCATTAACAGAAATCCGGCAAGGAGGAGGGTTTC |
| | | TTCTTGTGTCTACTGATATTGCAGCAAGGGGGATTGATCTACCGGAAAC |
| | | AAC[C]CACATCTTCAACTTTGATCTCCCACAGACAGCTACAGATTATCT |
| | | TCACCGAGCTGGAAGAGCTGGTCGAAAACCCTTTTCGGATAGGAAGTG |
| | | CATTGTTA |
| 42 | 6007107 | ATTCACGAGCAGCTTCATTAACAGAAATCCGGCAAGGAGGAGGGTTTC |
| | | TTCTTGTGTCTACTGATATTGCAGCAAGGGGGATTGATCTACCGGAAAC |
| | | AAC[T]CACATCTTCAACTTTGATCTCCCACAGACAGCTACAGATTATCT |
| | | TCACCGAGCTGGAAGAGCTGGTCGAAAACCCTTTTCGGATAGGAAGTG |
| | | CATTGTTA |
| 43 | 6058829 | CCACCGTCCTCCTAGGRCTAGCMAGCGCRAGCTTCCTCTTCCACGGCTC |
| | | CTTRAACGAAACATCAGGGATGGAGCCGCGCGTGGGGATTACGCGCCA |
| | | CGT[G]GGGATGAGATTAGCCACGACGAAGAGCAAATGCTCCAACGGC |
| | | CACGGCGGBTTGAACTTCCTGCTGATCCCRCACATGGCGCCGTTGAGG |
| | | AHGAGCCCGT |
| 44 | 6058829 | CCACCGTCCTCCTAGGRCTAGCMAGCGCRAGCTTCCTCTTCCACGGCTC |
| | | CTTRAACGAAACATCAGGGATGGAGCCGCGCGTGGGGATTACGCGCCA |
| | | CGT[A]GGGATGAGATTAGCCACGACGAAGAGCAAATGCTCCAACGGC |
| | | CACGGCGGBTTGAACTTCCTGCTGATCCCRCACATGGCGCCGTTGAGG |
| | | AHGAGCCCGT |

1. A Brassica oleracea plant wherein the plant resistant to the plant pathogen Albugo candida , and wherein the resistance is encoded by one genomic region located on chromosome 2 between base pairs 5373001 and 6058829 of the Brassica oleracea reference genome JZS v2.

2. The Brassica oleracea plant according to claim 1, wherein said genomic region is obtainable, obtained, or is from a Brassica oleracea plant deposited under deposit number NCIMB 43452.

3. The Brassica oleracea plant according to claim 1, wherein said plant comprises one or more genomic sequences selected from the group consisting of SEQ ID Nos. 1, SEQ) ID No. 3, SEQ ID No. 5, SEQ ID No. 7, SEQ ID NO. 9, SECLID No. 11, SEQ ID No. 13, SEQ ID No. 15, SEQ ID No. 17, SEQ ID No. 19, SEQ ID No 21, SEQ) ID No. 23, SEQ) ID No. 25, SEQ ID No. 27, SEQ ID No. 29, SEQ ID No. 31, SECLID No. 33, SEQ ID No. 35, SEQ ID No. 37, SEQ ID No. 39, SEQ ID No. 41, and SEO) ID No. 43.

4. The Brassica oleracea plant according to claim 1, wherein said plant is cytoplasmic male sterile (CMS).

5. The Brassica oleracea plant according to claim 1, said plant is a hybrid plant.

6. The Brassica oleracea plant according to claim 1, wherein said plant is a Brassica oleracea plant deposited under deposit number NCIMB 43452.

7. The Brassica oleracea plant according to claim 1, wherein the plant is selected from the group consisting of Brassica oleracea convar. botrytis var. botrytis, Brassica oleracea convar. botrytis var. cymosa, Brassica oleracea convar. botrytis var. asparagoides, Brassica oleracea convar. oleracea var. gemnifera, Brassica oleracea convar. capitata var. alba, Brassica oleracea convar. capitata var. rubra, Brassica oleracea convar. capitata var. sabauda, Brassica oleracea convar. acephela var. sabellica , Brassica oleracea convar. acephela var. gongylodes; and Brassica oleracea var. tronchuda syn. costata.

8. A method for identifying a genomically encoded resistance against the plant pathogen Albugo candida as found in Brassica oleracea plant deposited under deposit number NCIMB 43452, the method comprises the step of detecting one or more genomic sequences selected from the group consisting of SEQ ID Nos. 1, SEQ ID No. 3, SEQ. ID. No. 5, SEQ ID No 7, SEQ ID No 9, SEQ ID No 11, SEQ ID No. 13, SEQ ID No. 15, SEQ ID No. 17, SEQ ID No. 19, SEO ID No. 21, SEQ ID No. 23, SEQ ID No. 25, SEQ ID No. 27, SEQ ID No. 29, SEQ ID No 31, SEQ ID No. 33, SEQ ID No. 35, SEQ ID No. 37, SEQ ID No. 39, SEQ ID.No. 41, and SEQ ID No. 43.

9. A seed or plant part of the Brassica oleracea plant according to claim 1.

10. A good capable of providing the hybrid plant according to claim 5.

11. A molecular marker selected from the group consisting of SEQ ID Nos. 1, SEQ ID No. 3, SEQ ID No. 5, SEO ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37, SEQ ID No. 39, SEQ ID No. 41, and SEO ID No. 43.


SEQUENCE LISTING (44 sequences)

SEQ ID NO: 1 - DNA, 201 bp, Brassica oleracea
aaaaaatatggagtgaaatacaaagattaaattaataaatagaatgaaacaataaagattcagaccaaaacctatcaaccaactaagcaaccagacatgctmgaacmaraaaaatygrggatagtcgaagtcraraacaatgcahcacaataccgagarawaaktgttctcaaaccttgaaacaaytccttctacagcykc

SEQ ID NO: 2 - DNA, 201 bp, Brassica oleracea
aaaaaatatggagtgaaatacaaagattaaattaataaatagaatgaaacaataaagattcagaccaaaacctatcaaccaactaagcaaccagacatgccmgaacmaraaaaatygrggatagtcgaagtcraraacaatgcahcacaataccgagarawaaktgttctcaaaccttgaaacaaytccttctacagcykc

SEQ ID NO: 3 - DNA, 201 bp, Brassica oleracea
atcgaataatgtaatttgtatttttataaatttaatttcactcaatayayatatatatgatatagtcatatagacgtggyttggcagaaaaagakggagaacacactcatggttwatagaaaaagagggaacaaagtaatagcgaggttgtccywttcttcttgatcartgattatsratckgtttcgtagtgctcttgtt

SEQ ID NO: 4 - DNA, 201 bp, Brassica oleracea
atcgaataatgtaatttgtatttttataaatttaatttcactcaatayayatatatatgatatagtcatatagacgtggyttggcagaaaaagakggagagcacactcatggttwatagaaaaagagggaacaaagtaatagcgaggttgtccywttcttcttgatcartgattatsratckgtttcgtagtgctcttgtt

SEQ ID NO: 5 - DNA, 201 bp, Brassica oleracea
catatcataaaagctaatggaagtaaatgggaacsaaccatctscgagartcataaccagctatattggcgacaccctccaaagcttccctccatgcctttaccttttcttctttccccacaggttttttcaaaggctttcccgaaatctccggtctgcttcctaacmtcagatggatccacttcgtagaaaatggatatc

SEQ ID NO: 6 - DNA, 201 bp, Brassica oleracea
catatcataaaagctaatggaagtaaatgggaacsaaccatctscgagartcataaccagctatattggcgacaccctccaaagcttccctccatgccttgaccttttcttctttccccacaggttttttcaaaggctttcccgaaatctccggtctgcttcctaacmtcagatggatccacttcgtagaaaatggatatc

SEQ ID NO: 7 - DNA, 201 bp, Brassica oleracea
aaaaacaaatacaagaaatgtaccaactgttaagccaagaaatctgagaacacataatgtcagaggctcagagcacgagcacgagtatttcacataactacaagatggtgttaaaagatttaccaaaataaatgcatttggcatatacggaaggaataattagaaatacaaatctaagaaatttatttgagttramaaaaa

SEQ ID NO: 8 - DNA, 201 bp, Brassica oleracea
aaaaacaaatacaagaaatgtaccaactgttaagccaagaaatctgagaacacataatgtcagaggctcagagcacgagcacgagtatttcacataactagaagatggtgttaaaagatttaccaaaataaatgcatttggcatatacggaaggaataattagaaatacaaatctaagaaatttatttgagttramaaaaa

SEQ ID NO: 9 - DNA, 201 bp, Brassica oleracea
aacttgagttatttcattctcatgtactcgaacacatacatcttgagaactgaataatatagtataaacgaataaaactgaacttagggattgctcaaactgagtttcccacttcatcatgtgtggctcatagggcaagagcagagctaaggttcatagggttcatatacttggtggtaccggtcaatatatgacggacta

SEQ ID NO: 10 - DNA, 201 bp, Brassica oleracea
aacttgagttatttcattctcatgtactcgaacacatacatcttgagaactgaataatatagtataaacgaataaaactgaacttagggattgctcaaaccgagtttcccacttcatcatgtgtggctcatagggcaagagcagagctaaggttcatagggttcatatacttggtggtaccggtcaatatatgacggacta

SEQ ID NO: 11 - DNA, 401 bp, Brassica oleracea
acctcctcgctgatgaccttttcgagaatcatccaaggaggatgactctgtatgaactgacagtttctttccatgttgatgcaccgaaaacaagaagcaaccaaacaaaagaaagaagattgtaaaagtccattcrtacaccaagatcaaaccagtccatggcatgatttgcctcggcayaatcacaaaggaagttccaatggatatcagaagtgcagtaaaacagactagaactgaaactgcgcctaagcgctgaggaactttggagtgtatgctgccactgtggagttgatagctgggatacatggttgaaaatgtagaaacaccgcgtgttccattagatctgattctgtaataaagatatctaatctgattgaataatgaaccctcatgaacctgaa

SEQ ID NO: 12 - DNA, 401 bp, Brassica oleracea
acctcctcgctgatgaccttttcgagaatcatccaaggaggatgactctgtatgaactgacagtttctttccatgttgatgcaccgaaaacaagaagcaaccaaacaaaagaaagaagattgtaaaagtccattcrtacaccaagatcaaaccagtccatggcatgatttgcctcggcayaatcacaaaggaagttccaacggatatcagaagtgcagtaaaacagactagaactgaaactgcgcctaagcgctgaggaactttggagtgtatgctgccactgtggagttgatagctgggatacatggttgaaaatgtagaaacaccgcgtgttccattagatctgattctgtaataaagatatctaatctgattgaataatgaaccctcatgaacctgaa

SEQ ID NO: 13 - DNA, 401 bp, Brassica oleracea
tgtagtaacgtcacaagacacacctcctcgctgatgaccttttcgagaatcatccaaggaggatgactctgtatgaactgacagtttctttccatgttgatgcaccgaaaacaagaagcaaccaaacaaaagaaagaagattgtaaaagtccattcrtacaccaagatcaaaccagtccatggcatgatttgcctcggcacaatcacaaaggaagttccaayggatatcagaagtgcagtaaaacagactagaactgaaactgcgcctaagcgctgaggaactttggagtgtatgctgccactgtggagttgatagctgggatacatggttgaaaatgtagaaacaccgcgtgttccattagatctgattctgtaataaagatatctaatctgattgaata

SEQ ID NO: 14 - DNA, 401 bp, Brassica oleracea
tgtagtaacgtcacaagacacacctcctcgctgatgaccttttcgagaatcatccaaggaggatgactctgtatgaactgacagtttctttccatgttgatgcaccgaaaacaagaagcaaccaaacaaaagaaagaagattgtaaaagtccattcrtacaccaagatcaaaccagtccatggcatgatttgcctcggcataatcacaaaggaagttccaayggatatcagaagtgcagtaaaacagactagaactgaaactgcgcctaagcgctgaggaactttggagtgtatgctgccactgtggagttgatagctgggatacatggttgaaaatgtagaaacaccgcgtgttccattagatctgattctgtaataaagatatctaatctgattgaata

SEQ ID NO: 15 - DNA, 195 bp, Brassica oleracea
tcaagaacgaccatcccgttccgatcaagatgatcacggtgaaaagcaacacgacacgaatgaattggaagatgtagaagaggatgtcccatccgtgaggagtccccgtgatcttcacgtartgcttatcytcagctgcgcagatcagattcaaagacttgattaaaagcagacccgccatgaggagatggatcc

SEQ ID NO: 16 - DNA, 195 bp, Brassica oleracea
tcaagaacgaccatcccgttccgatcaagatgatcacggtgaaaagcaacacgacacgaatgaattggaagatgtagaagaggatgtcccatccgtgaggcgtccccgtgatcttcacgtartgcttatcytcagctgcgcagatcagattcaaagacttgattaaaagcagacccgccatgaggagatggatcc

SEQ ID NO: 17 - DNA, 201 bp, Brassica oleracea
gagatggagttggtgtggcatgactcagccaatggytcgagccgtcctacaaattcgaacaagacttcyacagactcagttagatggcctcaatggaagtgaaccaacmgagaagtgaatatgattacgtttccggttcagtggattaaccaacaggttgcagatcattgaatcgatatgtttgtatgtttaaatataata

SEQ ID NO: 18 - DNA, 201 bp, Brassica oleracea
gagatggagttggtgtggcatgactcagccaatggytcgagccgtcctacaaattcgaacaagacttcyacagactcagttagatggcctcaatggaagtaaaccaacmgagaagtgaatatgattacgtttccggttcagtggattaaccaacaggttgcagatcattgaatcgatatgtttgtatgtttaaatataata

SEQ ID NO: 19 - DNA, 201 bp, Brassica oleracea
gtttctataagaagaaaccagaagaagggtctattagtggaagggtccagaggcttgcdaagtatcgattcttgaagaaacaatcggatctkttgttgaattctgatgatttggctgctatgtggaattgtctgagagaaaattgtgtgattgatgatgccactggtgctgaaaagatgaactatgaagacttctgccaca

SEQ ID NO: 20 - DNA, 201 bp, Brassica oleracea
gtttctataagaagaaaccagaagaagggtctattagtggaagggtccagaggcttgcdaagtatcgattcttgaagaaacaatcggatctkttgttgaactctgatgatttggctgctatgtggaattgtctgagagaaaattgtgtgattgatgatgccactggtgctgaaaagatgaactatgaagacttctgccaca

SEQ ID NO: 21 - DNA, 305 bp, Brassica oleracea
tcacgcatgaccatgatattgttcctcatctgcctccttactacaaccattttcctcaaaaaacataccaccacttcccaacagaggtgtggctaagagatgtctgttccttgaatcatagtgtggagaaagtttgtgacaacaccggtgaagatccaacatgcagcaggtcggtgaagggcaatagcatttcagaccatctaaggtactttggggtagagttgcattgtgagacttggagacaatgctcaatagtgatgagccatgagatggatagattcagcaagaaggattcaaagggtaat

SEQ ID NO: 22 - DNA, 305 bp, Brassica oleracea
tcacgcatgaccatgatattgttcctcatctgcctccttactacaaccattttcctcaaaaaacataccaccacttcccaacagaggtgtggctaagagatgtcagttccttgaatcatagtgtggagaaagtttgtgacaacaccggtgaagatccaacatgcagcaggtcggtgaagggcaatagcatttcagaccatctaaggtactttggggtagagttgcattgtgagacttggagacaatgctcaatagtgatgagccatgagatggatagattcagcaagaaggattcaaagggtaat

SEQ ID NO: 23 - DNA, 401 bp, Brassica oleracea
catagtgtggagaaagtttgtgacaacaccggtgaagatccaacatgcagcaggtcggtgaagggcaatagcatttcagaccatctaaggtactttggggtagagttgcattgtgagacttggagacaatgctcaatagtgatgagccatgagatggatagattcagcaagaaggattcaaagggtaatctaatcatgtcacggaatgttccttccaccaacggtaacaaaacagaatctcttatcgaaaatggggatctttagtctataggaatcgttgattcaagtcttggtcaagcaaagcttgcttcaaaaggagattccggtgttggagaaagaaagaaagtgtatagatacatataatcaagactttgtaaataggttgtaggttgatagtacgt

SEQ ID NO: 24 - DNA, 401 bp, Brassica oleracea
catagtgtggagaaagtttgtgacaacaccggtgaagatccaacatgcagcaggtcggtgaagggcaatagcatttcagaccatctaaggtactttggggtagagttgcattgtgagacttggagacaatgctcaatagtgatgagccatgagatggatagattcagcaagaaggattcaaagggtaatctaatcatgtcgcggaatgttccttccaccaacggtaacaaaacagaatctcttatcgaaaatggggatctttagtctataggaatcgttgattcaagtcttggtcaagcaaagcttgcttcaaaaggagattccggtgttggagaaagaaagaaagtgtatagatacatataatcaagactttgtaaataggttgtaggttgatagtacgt

SEQ ID NO: 25 - DNA, 201 bp, Brassica oleracea
cctttgtactaaaccacttaatggcacagtgctcatgaacgagcctgaggtcacctttgcaactgcattccattttcaacgtgttgccttcctcgcagacatcaagacaaatcctgcacaccgcttcttcttcagggatctcttcttcagtttcttccgcagtaaccggagtgatttcatctccacaaccacttgcttcat

SEQ ID NO: 26 - DNA, 201 bp, Brassica oleracea
cctttgtactaaaccacttaatggcacagtgctcatgaacgagcctgaggtcacctttgcaactgcattccattttcaacgtgttgccttcctcgcagacgtcaagacaaatcctgcacaccgcttcttcttcagggatctcttcttcagtttcttccgcagtaaccggagtgatttcatctccacaaccacttgcttcat

SEQ ID NO: 27 - DNA, 201 bp, Brassica oleracea
ttaggtgtcaggtccygggttgtgaagtggatataagcgagctcaaagggtaycataraaggcatagggtttgyctcacgtgtgctaacgctagctccgtggtgcttgagggagtggataagagatactgtcaacagtgtggaaagtawgttccttttattgttaatttgatcctatgctttatggcttaacagatacata

SEQ ID NO: 28 - DNA, 201 bp, Brassica oleracea
ttaggtgtcaggtccygggttgtgaagtggatataagcgagctcaaagggtaycataraaggcatagggtttgyctcacgtgtgctaacgctagctccgttgtgcttgagggagtggataagagatactgtcaacagtgtggaaagtawgttccttttattgttaatttgatcctatgctttatggcttaacagatacata

SEQ ID NO: 29 - DNA, 201 bp, Brassica oleracea
tcaacagtctcaactctacggttcaaacacctgaatctcagtttgtgcaccggttgctcgacagactacatgctctccatcaggatcacatgagctacaaacatgtggttgaaaagccttttagttttccgcttcctaataargatgatcttgtctggtttttaaacaaacccttttaactgttgttccaggggatgttct

SEQ ID NO: 30 - DNA, 201 bp, Brassica oleracea
tcaacagtctcaactctacggttcaaacacctgaatctcagtttgtgcaccggttgctcgacagactacatgctctccatcaggatcacatgagctacaagcatgtggttgaaaagccttttagttttccgcttcctaataargatgatcttgtctggtttttaaacaaacccttttaactgttgttccaggggatgttct

SEQ ID NO: 31 - DNA, 201 bp, Brassica oleracea
aaccataatctggagamttttgaccaaaagcatattgacasaagatctgcagagcccaagttgaagctggaaatatcatctcatacatatggttggtccytagtcccagtgacttgagaagttttttatcttcggttgtaatgataacaatacttcccggaccaacccatccacgctggtttgccatctcctctaattgyc

SEQ ID NO: 32 - DNA, 201 bp, Brassica oleracea
aaccataatctggagamttttgaccaaaagcatattgacasaagatctgcagagcccaagttgaagctggaaatatcatctcatacatatggttggtccygagtcccagtgacttgagaagttttttatcttcggttgtaatgataacaatacttcccggaccaacccatccacgctggtttgccatctcctctaattgyc

SEQ ID NO: 33 - DNA, 201 bp, Brassica oleracea
tttgaattccacaagattagctatacaryattactttttgaaactaaactaagttatattgtaacgcatgacsggctacagytaatggactttccacgctgactcactckgttggtgtgcttcatatgcgtgcgcatggcggtatattaattttttggaggctcctargacttgtytattaactcttaatcaaccacrtra

SEQ ID NO: 34 - DNA, 201 bp, Brassica oleracea
tttgaattccacaagattagctatacaryattactttttgaaactaaactaagttatattgtaacgcatgacsggctacagytaatggactttccacgctcactcactckgttggtgtgcttcatatgcgtgcgcatggcggtatattaattttttggaggctcctargacttgtytattaactcttaatcaaccacrtra

SEQ ID NO: 35 - DNA, 201 bp, Brassica oleracea
cgaggagttgtacttttttctttgtaaacaatatttgcttgcgcaataaattgaacattcccgaaaataacctatcgcttttacccctaaaaaaaattaccgccaaaaagttgaagcatgacatatttaggtccgagtcttcttcttcgtctcaatatatattgtggggccagcaatttggtgggaaccgtcgacgtggaa

SEQ ID NO: 36 - DNA, 201 bp, Brassica oleracea
cgaggagttgtacttttttctttgtaaacaatatttgcttgcgcaataaattgaacattcccgaaaataacctatcgcttttacccctaaaaaaaattactgccaaaaagttgaagcatgacatatttaggtccgagtcttcttcttcgtctcaatatatattgtggggccagcaatttggtgggaaccgtcgacgtggaa

SEQ ID NO: 37 - DNA, 201 bp, Brassica oleracea
accccaacacattgccttgatgttgaaattaattaatcactatccgtgttcartattgtctctccagscaagtaagtatttgattttaatcatactttaaatttacaytgctcttggccgcctagaagaaacataacaattcaggcctttgatcttgaccycgttcgaaaataggctcttctgctgtgaaccaaaggagta

SEQ ID NO: 38 - DNA, 201 bp, Brassica oleracea
accccaacacattgccttgatgttgaaattaattaatcactatccgtgttcartattgtctctccagscaagtaagtatttgattttaatcatactttaagtttacaytgctcttggccgcctagaagaaacataacaattcaggcctttgatcttgaccycgttcgaaaataggctcttctgctgtgaaccaaaggagta

SEQ ID NO: 39 - DNA, 201 bp, Brassica oleracea
tgcctcgatcttgacatrarctatattgatgtctgtcagattctttgtgtattcatctgtctycttargctcaccaatcaacccagsagcraagcttmgaacttcaaggctacgcaagttgagaggaagaccaatcaagtgagcccacakagggatcgactccatatctggagtggaggcctcgtgcttggaggtcaacgr

SEQ ID NO: 40 - DNA, 201 bp, Brassica oleracea
tgcctcgatcttgacatrarctatattgatgtctgtcagattctttgtgtattcatctgtctycttargctcaccaatcaacccagsagcraagcttmgaccttcaaggctacgcaagttgagaggaagaccaatcaagtgagcccacakagggatcgactccatatctggagtggaggcctcgtgcttggaggtcaacgr

SEQ ID NO: 41 - DNA, 201 bp, Brassica oleracea
attcacgagcagcttcattaacagaaatccggcaaggaggagggtttcttcttgtgtctactgatattgcagcaagggggattgatctaccggaaacaacccacatcttcaactttgatctcccacagacagctacagattatcttcaccgagctggaagagctggtcgaaaacccttttcggataggaagtgcattgtta

SEQ ID NO: 42 - DNA, 201 bp, Brassica oleracea
attcacgagcagcttcattaacagaaatccggcaaggaggagggtttcttcttgtgtctactgatattgcagcaagggggattgatctaccggaaacaactcacatcttcaactttgatctcccacagacagctacagattatcttcaccgagctggaagagctggtcgaaaacccttttcggataggaagtgcattgtta

SEQ ID NO: 43 - DNA, 201 bp, Brassica oleracea
ccaccgtcctcctaggrctagcmagcgcragcttcctcttccacggctccttraacgaaacatcagggatggagccgcgcgtggggattacgcgccacgtggggatgagattagccacgacgaagagcaaatgctccaacggccacggcggbttgaacttcctgctgatcccrcacatggcgccgttgaggahgagcccgt

SEQ ID NO: 44 - DNA, 201 bp, Brassica oleracea
ccaccgtcctcctaggrctagcmagcgcragcttcctcttccacggctccttraacgaaacatcagggatggagccgcgcgtggggattacgcgccacgtagggatgagattagccacgacgaagagcaaatgctccaacggccacggcggbttgaacttcctgctgatcccrcacatggcgccgttgaggahgagcccgt